(19) United States
(12) Patent Application Publication
(10) Pub. No.: US 2022/0265265 A1
(43) Pub. Date: Aug. 25, 2022

(54) MENISCUS SUTURE DEVICE

(71) Applicants: José Leonardo Rocha DE FARIA, Rio de Janeiro RJ (BR); Cristiano Siqueira PEREIRA, Juiz de Fora MG (BR); José Alencar MARQUES JUNIOR, Juiz de Fora MG (BR)

(72) Inventors: José Leonardo Rocha DE FARIA, Rio de Janeiro RJ (BR); Cristiano Siqueira PEREIRA, Juiz de Fora MG (BR)

(21) Appl. No.: 17/678,273

(22) Filed: Feb. 23, 2022

(30) Foreign Application Priority Data
Feb. 23, 2021 (BR) BR102021003388-6

(51) Int. Cl. A61B 17/04; A61B 17/06

(52) U.S. Cl. CPC A61B 17/0483; A61B 17/06109; A61B 2017/06042; A61B 2017/00367

(57) ABSTRACT

The present invention refers to a meniscus suture device comprising a handle for handling the device which comprises a push button with axial displacement, a cannula extending from the handle and having a free extremity opposite the handle, a movable rod housed and axially displaceable inside the cannula, the movable rod comprising a first extremity which extends inside the handle and a second extremity, opposite the handle, which has an indenture for capturing a suture thread, wherein the first extremity of the rod comprises a driving mechanism connected to the push button of the handle, for activating the axial displacement of the movable rod inside the cannula, and whereby when the axial displacement of the movable rod inside the cannula is activated, the second extremity of the rod extends outside the orifice of the cannula.

The present invention refers to a meniscus suture device to assist in meniscus lesions repair surgeries, comprising a retractable hook in the extremity thereof to retain the suture, providing more ease to the operation.

### DESCRIPTION OF THE STATE OF THE ART

Different devices used in surgical procedures for retaining the suture thread are already known to the state of the art.

Document WO2015/089034 discloses a device to assist in holding a portion of a suture during an intra-abdominal suturing procedure, the device including an elongated tubular body, a handle, an inner member element, and a push button. The inner member element may include at least one grasping element that expands as the at least one grasping element is extended beyond a distal end of the elongated tubular. The device may include a proximal spring disposed within the push button and abutting against a proximal end of the inner member element to allow the at least one grasping element to retract into the elongated tubular body in a proximal direction.

Document U.S. Pat. No. 5,618,290 discloses an endoscopic suturing device provided for manipulating a suture to form a stitch through a tissue within a body cavity. The suturing device comprises a needle with a rod, a pointed head located at the distal end of the needle rod, a hook formed proximally to the pointed head, and a latch for opening and closing the hook. The latch is biased to the open position. A tubular sheath is provided which is concentric with the needle rod, the needle rod being extensible within the tubular sheath, such that the hook expands beyond the diameter of the tubular sheath when the needle rod is extended. The suture is clamped by the device by inserting the suture into the hook and sliding the tubular sheath over the latch, thereby closing the hook. The suture can then be released by sliding the tubular sheath to uncover the latch, thereby opening the hook.

Both the above cited documents present suturing devices with means to capture the suture thread which, when activated, increase the thickness of the extremity of the device, that is, the point for capturing the thread extends beyond the diameter of the device. These suture devices are destined for use in the abdominal region, where the tissues are softer, with less resistance, and have a greater distance than the tissues of the knee articulation which present higher resistance and less spacing. It is important to observe that the articulation of the knee, in the medial and lateral tibial femur region, presents an opening between the bone surfaces which varies from 4 to 6 mm, therefore, an increase in thickness during the procedure increases considerably the risk of undesired lesions to the tissue, when the suture device penetrates this region.

Additionally, the suture device of document U.S. Pat. No. 5,618,290 extends frontwards to release the hook thereof to capture the thread, which leads to an excessive elevation of the device in the patient's tissue.

### Objectives of the Invention

The present invention has as objective to provide a meniscus suture device with a mechanism that is adapted to capture the suture thread with sufficiently high resistance for meniscus surgeries and without increasing the thickness thereof.

Another objective of the invention is to minimize the risk of lesions resulting from the operation of the device in the patient.

### BRIEF DESCRIPTION OF THE INVENTION

The objectives of the invention are reached by means of a meniscus suture device comprising a handle for handling the device with an axial displacement push button and a cannula which extends from the handle having a free extremity opposite the handle. The device further comprises a movable rod housed and axially displaceable inside the cannula, having a first extremity which extends inside the handle, and a second extremity, opposite the handle, which comprises an indenture for capturing the suture thread, wherein the first extremity of the rod has a push device connected to the push button of the handle to drive the axial displacement of the movable rod within the cannula, whereby when the axial displacement of the movable rod is activated, the second extremity of the rod extends out of the orifice of the cannula.

Optionally, the extremity of the movable rod opposite the handle has a rounded shape and the indenture at the end of the movable rod has a hooked shape.

Alternatively, the free extremity of the cannula is beveled so that when the second extremity of the rod and the indenture are positioned in this beveled region of the free extremity of the cannula, then the movable rod is axially dislocated inside the cannula. Further, the beveled extremity can be tapered and have a pair of recesses in the transverse direction thereof.

In an alternative embodiment of the invention, the thickness of the cannula of the device comprises within 1 mm and 2 mm and the driving mechanism is a spring driving mechanism.

### BRIEF DESCRIPTION OF THE FIGURES

For a better understanding, the characteristics and advantages of the present invention will be presented and described together with their respective figures, which illustrate:

FIG. 1 is a front view of a preferred embodiment of the meniscus suture device according to the invention;

FIG. 2 is a side view of the meniscus suture device Illustrated in FIG. 1;

FIG. 3 is an enlarged end view of the cannula of the meniscus suture device shown in FIG. 2;

FIG. 4 is an enlarged view of the extremity of the cannula shown in FIG. 2 showing the rod;

FIG. 5 is a front view of the movable rod with the driving mechanism of the suture device in accordance with the preferred embodiment of the invention.

### DETAILED DESCRIPTION OF THE INVENTION

The meniscus suture device, object of the present invention, is used in repair surgeries (suture) of the meniscus, preferably by arthroscopy.

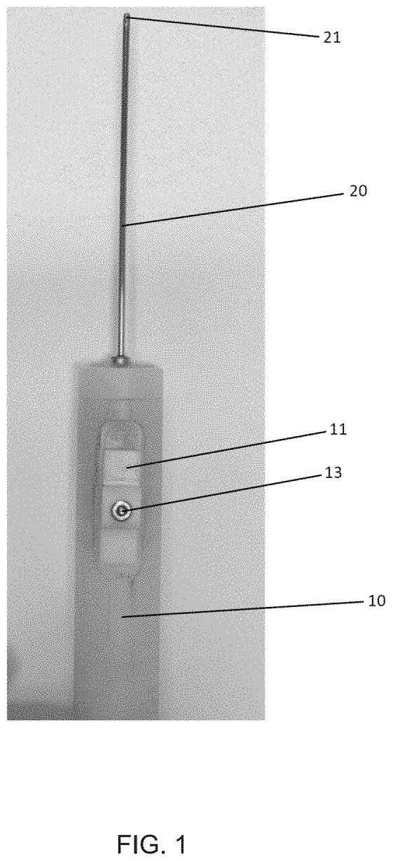

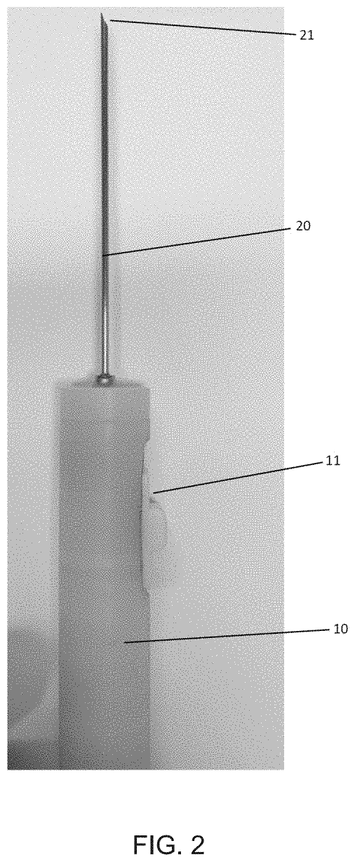

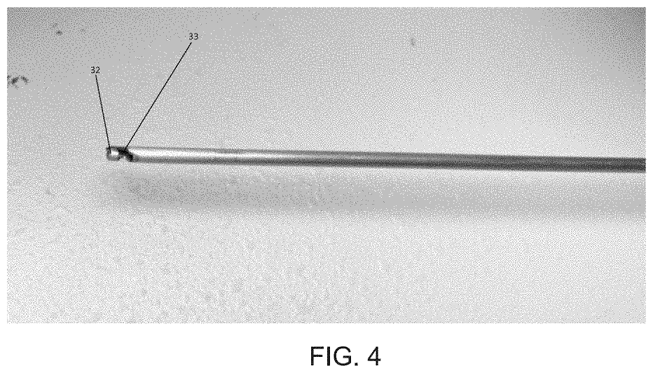

As can be seen in the embodiment of the invention shown in FIGS. 1 to 5, the meniscus suture device comprises a handle 10 for handling of the device by the surgeon having a push button 11 slidable in the axial direction of the handle 10.

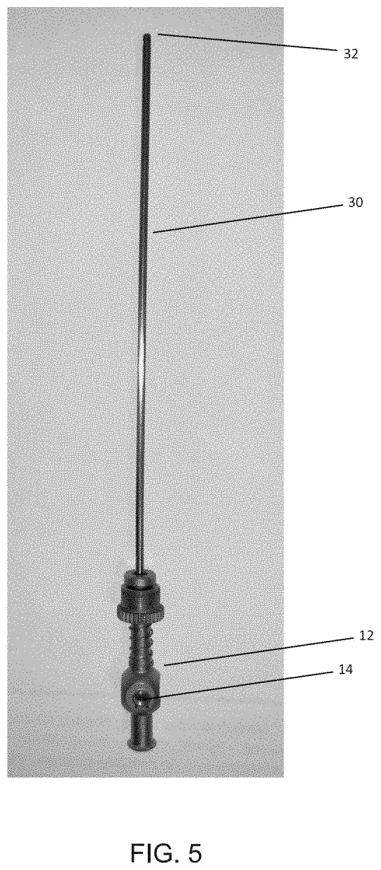

The device further comprises a cannula 20 which extends from the handle and having a free extremity 21 opposite the handle. Inside the cannula 20 there is a movable rod 30 having a larger extension than the cannula 20. As can be seen in FIG. 5, in a first extremity, the movable rod 30 extends into the handle 10, having a driving mechanism 20 which connects to the push button 11 of the handle 10.

This connection between the driving mechanism 12 and the push button 11 allows that, by means of the button 11, the movable rod 30 is axially dislocated inside the cannula 20 until a second extremity 32 reaches the free extremity 21 of the cannula 20. Preferably, this connection between the driving mechanism 12 and the push button 14 is made by means of a screw 13 shown in FIG. 1 which is threaded on the female screw thread 14 located in the driving mechanism 12, as illustrated in FIG. 5. In this manner, the movable rod 30 remains fixed in rotation, preventing the movable rod 30 from spinning around its axial axis inside the cannula 20 when activated.

This second extremity 32 of the rod has an indenture 33 used to capture the suture thread during a suture procedure. Thus, when the movable rod 30 is axially dislocated inside the cannula 20, the second extremity 32 of the rod 30 and the indenture 33 extend outside the orifice of the cannula 20, being able to capture the suture thread. In this manner, during the capture of the suture thread, there is no increase in the thickness of the suture device, since the second extremity, 32 with the indenture 33 present a smaller diameter than that of the cannula 20 and do not protrude radially in relation to this cannula 20.

In a preferred embodiment of the invention, the extremity of the rod 32 opposite the handle has a rounded shape to avoid lesions to the tissues. In this manner, when the movable rod 30 is axially dislocated, there is no risk of injuring or accidently puncturing the patient in case the extremity of the rod 32 contacts a tissue.

Preferably, the free extremity 21 of the cannula 20 is beveled, so that, when the movable rod 30 dislocates axially inside the cannula, the second extremity 32 of the rod and the indenture 33 remain in this beveled region of the free extremity 21 of the cannula 20. This construct is particularly advantageous, since it allows the indenture 33 to remain exposed outside the orifice of the cannula 20, without increasing the overall length of the device, avoiding possible damage to the patient's knee.

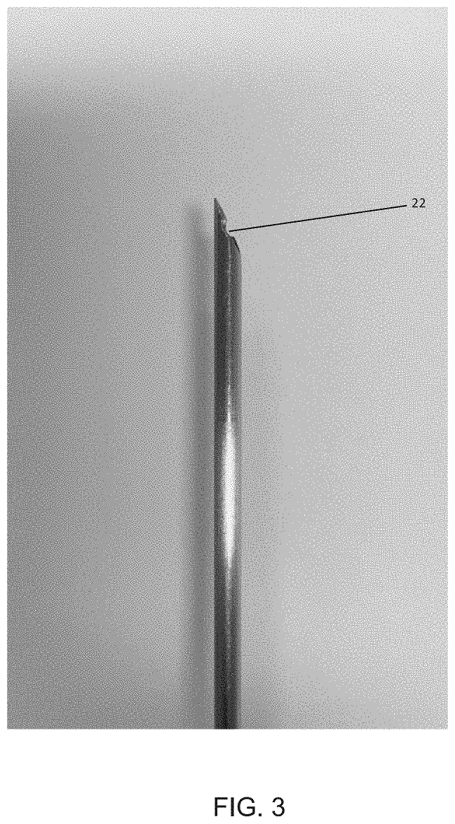

In an embodiment of the invention, the beveled free end 21 of the cannula 20 is also tapered, functioning additionally as a needle for executing stitches in a suture. The beveled free extremity 21 can, optionally, present a pair of small recesses 22 in the transversal section thereof, as shown in FIG. 3, which facilitate the positioning and adherence of the suture thread during the capture of the same.

The indenture 33 of the rod 30 is, preferably, a hook shaped indenture. This shape is used, since it facilitates the capture of the thread by the surgeon and guarantees that the same remains fixed in the indenture 33.

FIG. 5 shows an embodiment of the movable rod 30, wherein the driving mechanism 12 is a spring driving mechanism with an orifice wherein the push button 11 is threaded. When the button 11 is activated, the mechanism compresses the spring and dislocates the rod 30 in the direction of the move. With the free extremity of the rod 30 exposed, the surgeon can position indenture 33 around the suture thread, and then once again retract the rod 30. In this manner, the suture thread is firmly gathered into the cannula together with the rod 30, thus remaining safe and without risk of being lost.

The thickness of the cannula 20 is, preferably, comprised between 1 mm and 2 mm. This is due to the need to have a compact device which can be easily manipulated inside a small space such as the articulation of the knee, which presents an opening which varies from 4 to 6 mm, without impairing the use thereof and without the patient suffering with the movement of the same.

The present invention as described above allows that the execution of outside-in type sutures in small spaces, as is the case of surgeries of the meniscus, be carried out more easily by the surgeon and with less risks to the patient. The device according to the invention eliminates the need for the surgeon to “fish” the line with common tweezers or with the hand, providing more safety and agility during a delicate procedure.

Further, the suturing device according to the present invention can be used both as an instrument for the execution of the stitches themselves, as an auxiliary device just for controlling the suture thread, according to the need at the time.

Having described an example of preferred embodiment, it must be understood that the scope of the present invention covers other possible variations, not being limited solely by the content of the attached claims, being included therein the possible equivalents.

What is claimed is:

1. A meniscus suture device, comprising:
a handle for handling the device, whereby the handle comprises a push button with axial displacement;
a cannula extending from the handle and having a free end opposite the handle;
a movable rod housed and axially displaceable inside the cannula, the movable rod comprising a first extremity extending inside the handle and a second extremity, opposite to the handle, which has an indenture to capture a suture thread;
wherein the first extremity of the rod comprises a driving mechanism connected to the push button of the handle for activating the axial displacement of the movable rod inside the cannula, and
wherein, when the axial displacement of the movable rod inside the cannula is activated, the second extremity of the rod extends outside the orifice of the cannula.

2. The device according to claim 1, wherein the second extremity of the rod opposite the handle has a rounded shape.

3. The device according to claim 1, wherein the indenture in the second extremity of the rod is hook-shaped.

4. The device according to claim 1, wherein the free end of the cannula is beveled, and the second extremity of the rod and the indenture remain positioned in this beveled region of the free end of the cannula during the driving of the axial displacement of the movable rod inside the cannula.

5. The device, according to claim 4, wherein the beveled free end of the cannula is tapered.

6. The device, according to claim 4, wherein the beveled free end of the cannula has a pair of recesses in the transversal section thereof.

7. The device according to claim 1, wherein the cannula has a thickness of between 1 mm and 2 mm.

8. The device according to claim 1, wherein the driving mechanism is a spring driving mechanism.

9. The device according to claim 1, wherein the driving mechanism is fixed to the push button by a screw threaded on a female screw thread of the driving mechanism.
